(19) United States
(12) Patent Application Publication
(10) Pub. No.: US 2017/0337333 A1
(43) Pub. Date: Nov. 23, 2017

(54) Web-Based Data Analysis and Reporting System Advising a Health Care Provider

(71) Applicant: Darice M. Lulinski Grzybowski, JR., La Grange Park, IL (US)

(72) Inventor: Darice M. Lulinski Grzybowski, JR., La Grange Park, IL (US)

(21) Appl. No.: 15/474,641

(22) Filed: Mar. 30, 2017

Related U.S. Application Data
(63) Continuation-in-part of application No. 13/161,873, filed on Jun. 16, 2011, now abandoned.
(60) Provisional application No. 61/355,490, filed on Jun. 16, 2010.

(51) Int. Cl. G06F 19/00; G06Q 50/22

(52) U.S. Cl. CPC G06F 19/327; G06Q 50/22

(57) ABSTRACT

A web-based hospital data error analysis system which analyzes numerous hospital records recorded over a predetermined period of time for many different types of data entry errors. Results can be correlated according to department, physician, nurse or other data entry personnel.

This is a continuation-in-part of application number 13/161,873 filed Oct. 17, 201 which claimed priority from U.S. Provisional Patent Application No. 61/355,490 filed Jun. 16, 2010. applications Ser. No. 13/161,873 and 61/355,490 are hereby incorporated by reference in their entireties.

### BACKGROUND

### Field of the Invention

The present invention relates to management of an organization such as a health care provider and more particularly to a web-based data collection and analysis system that provides narrative and graphical interpretations of organization practices and procedures as compared to industry best practices on both a quantitative (objective) and qualitative (subjective) scale of measurement

### Description of the Prior Art

Management of a large organization like a hospital involves optimizing the actions and procedures of a large number of people so that these actions are coordinated, and so that costs can be minimized. Prior art methods have allowed collecting of metrics (data) within departments, and in specialty operations, to check efficiency of various procedures and processes and provide feedback that allows for improvements. However, many times, even if isolated departments are operating efficiently, the entire organization may be operating sub-optimum. This can be caused by the output of one department not meshing or synchronizing with the input requirements of another department. For example, in a hospital setting, just because a laboratory can turn around certain types of tests very quickly does not mean that the entire organization is optimal if providing test specimens or evaluating test results cannot keep up due to delays in managing the health information reports. Also, if proper data is not entered or maintained, billing records may become out of compliance or revenue losses may occur.

Traditionally, health care organizations employ consultants, at significant expense, to provide onsite observation and analysis of data. Or, as an alternative, they provide generic comparisons of data that is minimally ‘scraped’ from an existing information database. HIMetrix, as a unique method, does not rely on existing system data, and minimizes onsite consulting time, replacing it instead with an intelligent data collection methodology paired with consultative logic, to provide personalize business intelligence (data key indicators) to a site via web based access. By providing a method of continual monitoring, health care facilities can adjust and measure their performance over time. Utilizing experiential and published best practice research, proprietary formulas have been used to develop predictors of performance. These predictors will result in the recommendations provided to the clients in the online reporting module.

It would be advantageous to have a data collection and tracking system that could provide periodic, controlled snapshots of key operational indicators for an entire organization. This system could measure best practice variances across time to uncover hidden problems or process issues that lead to poor performance and thus reveal opportunities for decreased cost and optimized revenue.

### SUMMARY OF THE INVENTION

The present invention relates to a web-based data analysis and tracking system and method for a medium to large organization. Metrics can be determined by asking pre-defined and special questions designed and modified by the facility or organization. The totality of answers to these questions represent a set of metrics that can be weighted and analyzed to produce comparisons with standard or chosen thresholds and/or industry standards. Compliance with best practices can be evaluated, and variances from best practices can be flagged and made visible. The present invention first assures that the organization has reliable data. Next, it analyzes this data to form comparisons and usable output. Finally, it presents the output in various graphical formats that can be used to base management decisions upon such as dashboards, graphs such as bar graphs, pie charts or other types of graphs, and textual output.

The present invention includes a data and document Integrity module using heuristics based on studying tens of thousands of electronic and paper health records to determine the level of impact of documentation errors within the medical record. It allows facilities to create a baseline score via a specialized dashboard in which the software converts elements of documentation quality (accuracy, presence, and relationships) into impact scores for the healthcare organization. The facility can then drill down to individual providers (physicians and other staff) to help them evaluate changes in documentation necessary within the electronic health record.

### DESCRIPTION OF THE FIGURES

Attention is directed to several drawings that illustrate features of the present invention:

FIG. 1 is a flow chart of the general operation of the invention.

FIG. 2 shows one page of a sample questionnaire that can be used to collect metrics.

FIG. 3 shows a flow chart of an embodiment of an analysis module.

FIG. 4 shows a dashboard produced by the invention

FIG. 5 shows a bar chart produced by the invention.

FIG. 6 shows a pie chart produced by the invention.

FIG. 7 shows a physical diagram of an embodiment of the invention.

FIG. 8 shows a chart that presents documentation errors.

Several drawings and illustrations have been presented to aid in understanding the present invention. The scope of the present invention is not limited to what is shown in the figures.

### DESCRIPTION OF THE INVENTION

The present invention relates to a web-based data analysis and tracking system and method for a medium to large organization. In general, managers need to know what is happening in their organizations to make intelligent management decisions. To obtain this knowledge, it is necessary to collect data or metrics within various departments. Good metrics require specific questions with answers that can be frequently updated. In embodiments of the present invention, clients answer a series of “best practice” questions. Best practice questions are questions that probe facility practice to see if it meets best practice standards. Questions can be supplied in a survey-style of software that can be accessed over a network from a remote server. Client interviews and on-site visits can also provide valuable input data. Any way of gathering input data is within the scope of the present invention. Responses to the various questions are reduced and compiled to produce graphical and text output that shows variations from standards or best practice.

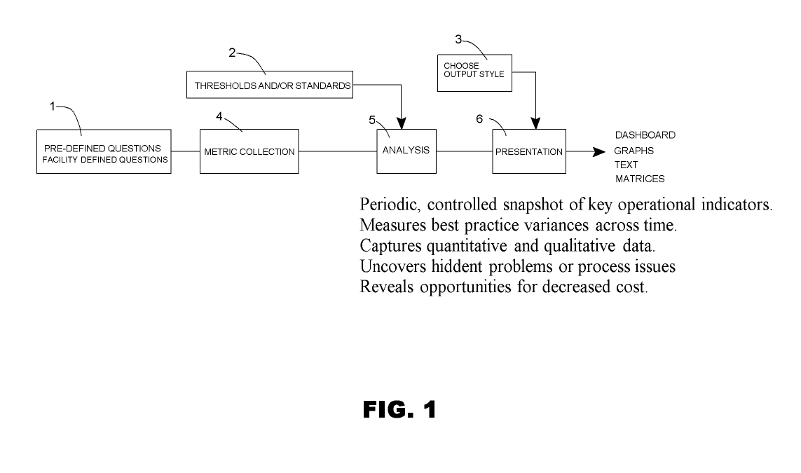

FIG. 1 shows a block diagram of an embodiment of the present invention. Two types of questions 1 can be ask, pre-defined questions and questions defined by the organization itself. Pre-defined questions represent a baseline set of fundamental best practice questions that are determined by the type of organization (hospital, manufacturer, etc.). In addition to baseline pre-defined questions, an organization or department can generate additional questions that relate more to the specific organization being analyzed. An example of a baseline question for a hospital might be: What is the total number of inpatient discharges annually excluding OB, newborn and pediatrics? A facility-defined question for this same hospital might be: What is the total number of annual inpatient discharges for home health care? Since not all hospitals have home health care departments, this is a narrower question that could relate to a particular hospital.

After a set of questions 1 is defined, the questions must be answered by facility personnel for metric collection 4. Metric collection 4 is simply the sorting, arranging and storing of the answers in a data base where an analysis module can 5 can operate upon the data.

In order to analyze the metrics, a set of thresholds and/or standards 2 must be supplied and is provided from a combination of the HIMentors team of experts research library, from publicly available data, and from the client organization itself. These can be common industry standards, or they can be at least partially specifically designed for the facility being analyzed. Industry standards can be metric values that represent “best practice” as agreed upon by the industry as a whole through industry and/or professional organizations as well as standards setting committees or the government. Thresholds and standards set by the particular facility represent where they would like to be. All of these thresholds and standards 2 can be modified as requirements change or if there is a realization that some threshold is too stringent or not stringent enough, or if industry or government standards are changed by the bodies that create and maintain them.

After metrics are collected 4 and thresholds and standards 2 are available, the metrics can be analyzed 5 against the thresholds and/or standards. Output format and style 3 can be standardized or chosen by the facility client to suit their needs. Generally, results are presented 6 in the form of graphics such as dashboards, bar graphs, pie charts and by any other presentation format or means. Clients can access both the survey and the output reports and graphics directly or over a network such as the World Wide Web. The survey and reports can be located anywhere in the network, and in particular on a server such as a data warehouse server(s). A cloud computing type model can be used with the various parts of the software accessible from remote servers in the network.

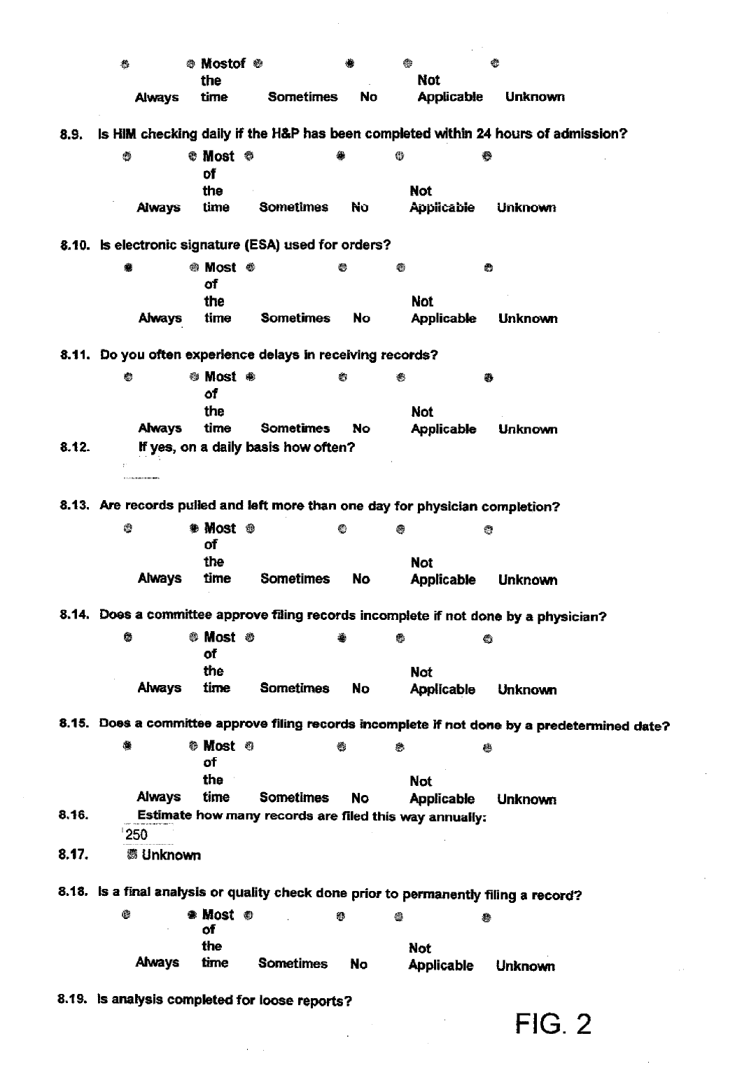

FIG. 2 shows a particular page from a questionnaire used to collect metrics. A sample of a full questionnaire is presented in the appendix to this description. Numerous questions appear that can be answered by choosing a point on a sliding scale such as: Always, Most of the Time, Sometimes, No or Never, NA or Unknown. As can be seen from FIG. 2, the metric data set that results from a complete answering of all of the questions in the questionnaire is a matrix of data points.

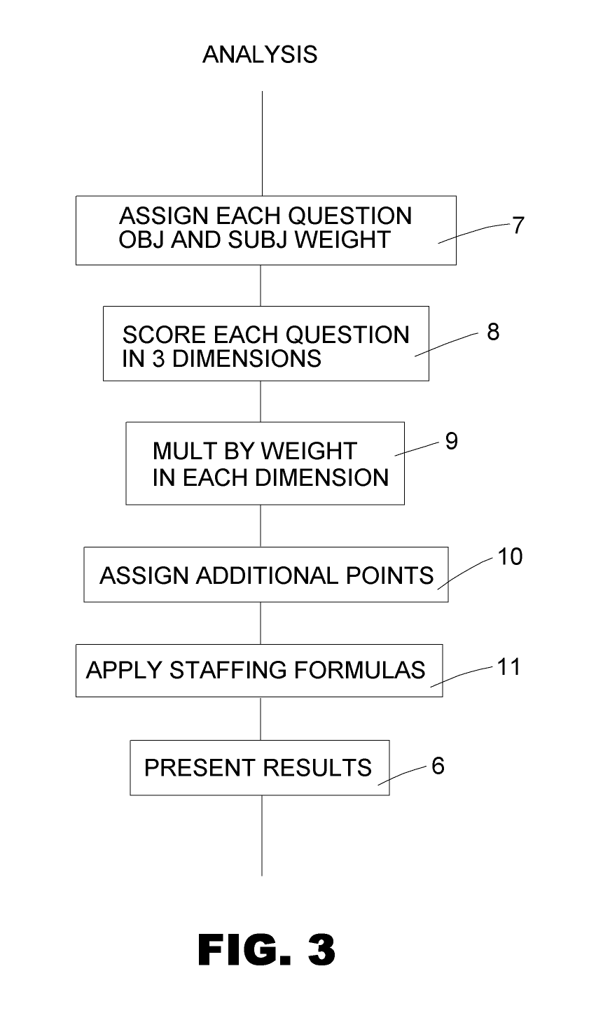

FIG. 3 shows a step-by-step breakdown of a particular embodiment of an analysis module. In this embodiment, each question is assigned an objective and subjective weight 7. Each response to a question is scored 8 in at least three dimensions: compliance, financial and operational based on a scale based on best practices throughout the industry and the supplied thresholds and standards 2. While three dimensions is preferred, any number of data dimensions may be used. Dimensional choice is generally guided by empirical practice gathered from years of hospital or organization management. Each question is multiplied 9 by its weight(s) within each of the dimensions to form a set of weighted metrics. Additional points 10 may be assigned based on specific knowledge within the industry. Additional formulas 11 may be applied to predict staffing resources. Finally, a graphical presentation 6 is made based on user choices.

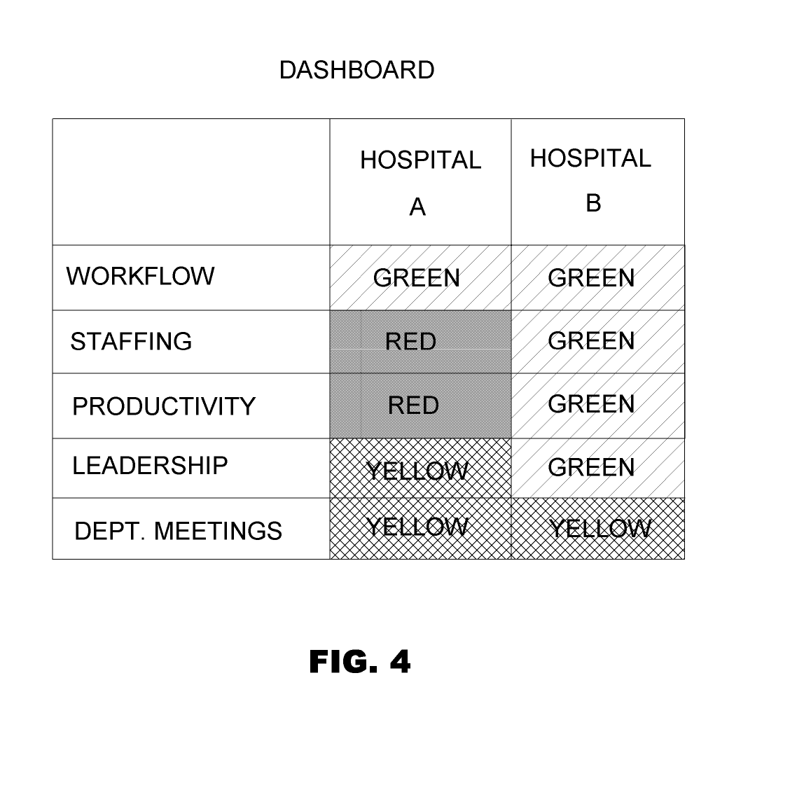

A common graphical output is a dashboard shown in FIG. 4. The dashboard can indicate scores of Red, Yellow or Green dependant on the values scored based on specific answers. Red can indicate an area of concern; Yellow can indicate that further investigation may be needed, while Green can indicate an acceptable or best level of practice.

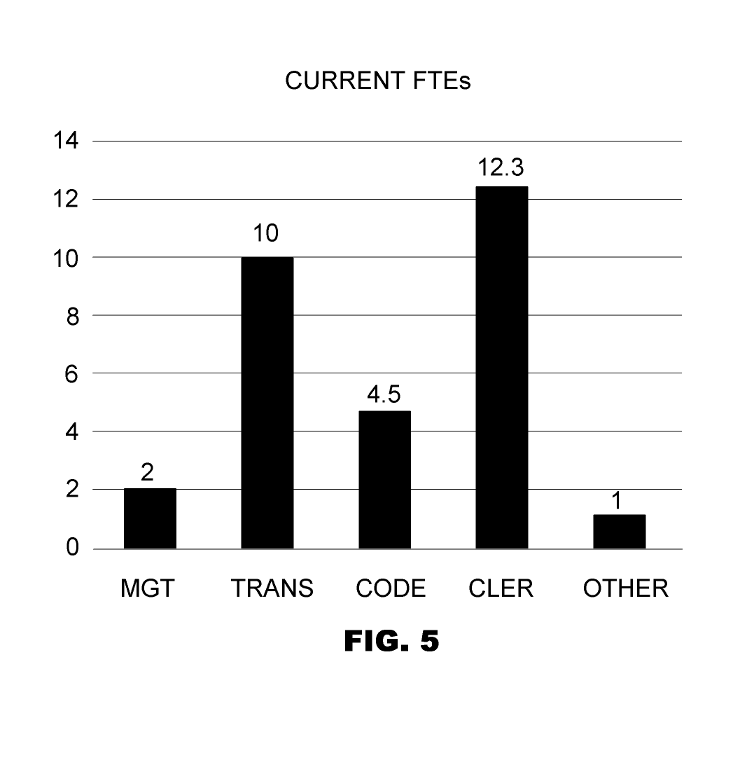

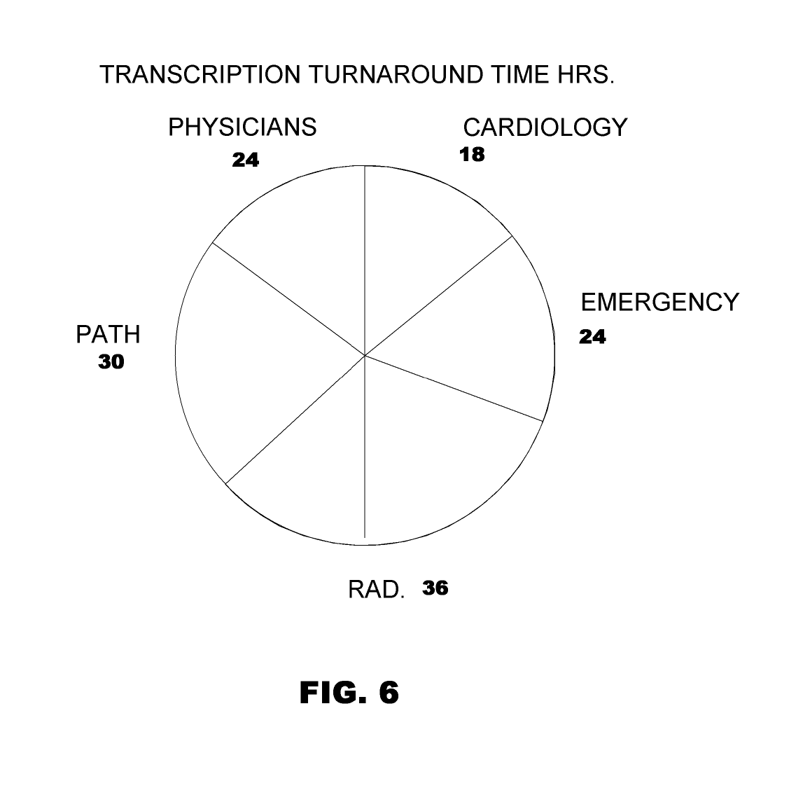

FIG. 5 shows a sample bar graph showing current full time employees (FTEs). FIG. 6 shows a pie chart for transcription turnaround times.

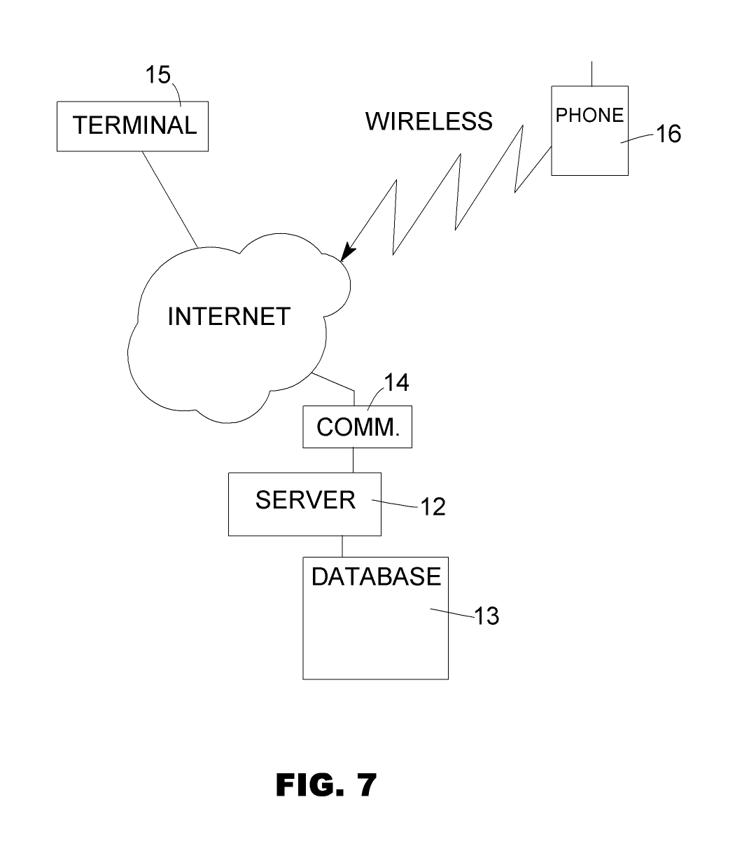

FIG. 7 shows an embodiment of a physical layout for the web-enabled system. A server 12 communicates with one or more databases 13 that are used to store metrics and results. The server 12 can be located remotely from the database 13. All communication can be over a network of any type. A web-interface 14 allows the server 12 to communicate over the Internet with remote terminals 15 where metrics can be entered, and results can be displayed. Metrics can also be entered and results displayed using devices like cellular telephones, palm devices, pad devices and the like 16. Each terminal, telephone and server also contains an internal processor, memories and communication electronic modules.

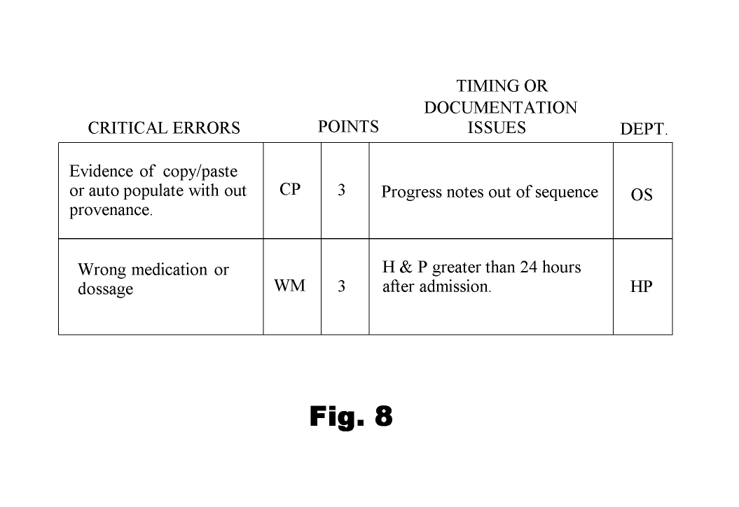

The present invention includes a data and document Integrity module using heuristics based on studying tens of thousands of electronic and paper health records to determine the level of impact of documentation errors within the medical record. It allows facilities to create a baseline score via a specialized dashboard in which the software converts elements of documentation quality (accuracy, presence, and relationships) into impact scores for the healthcare organization. The facility can then drill down to individual providers (physicians and other staff) to help them evaluate changes in documentation necessary within the electronic health record. FIG. 8 shows a presentation of two data errors, progress notes out of sequence, and an entry that was made later than 24 hours after the event. A weighted score is shown in the same row as each error. The weight is related to the severity of the error. These two entries are exemplary, the present invention finds numerous different types of documentation errors and reports them.

Several descriptions and illustrations have been presented to aid in understanding the features of the present invention. One skilled in the art will realize that numerous changes and variations are possible without departing from the spirit of the invention. Each of these changes and variations is within the scope of the present invention.

We claim:

1. A web-based method for locating and weighting errors in hospital data records comprising:
supplying a plurality of hospital records spanning a predetermined time period;
analyzing each record in relation to other supplied records to determine data collection or data entry errors;
weighting each data entry error with a weight according to a set of predetermined weights relating to severity of the data entry error;
correlating each data entry error according to department, physician, nurse or other data entry personnel;
presenting a display showing each error along with its weight;
presenting a visual display of data entry errors related to departments and/or particular hospital personnel.

2. The method of claim 1 wherein said visual display includes at least one of a dashboard, a bar graph, a pie chart or textual output.

3. The method of claim 1 wherein said visual display also shows deviations from best practice.

4. The method of claim 3 wherein said deviations are presented in graphical and text format.

5. The method of claim 3 wherein thresholds and/or standards are used to generate said deviations.

6. The method of claim 1 wherein clients can access both survey and output reports and graphics over said network.
